(19) United States
(12) Patent Application Publication
(10) Pub. No.: US 2010/0204083 A1
(43) Pub. Date: Aug. 12, 2010

(54) PROCESS FOR THE PREPARATION OF ETHYLGERANONITRILE

(75) Inventors: Martine Weis, Mannheim (DE); Bernhard Brunner, Heidelberg (DE); Klaus Ebel, Lampertheim (DE); Wolfgang Krause, Bruhl-Rohrhof (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/670,689

(22) PCT Filed: Jul. 16, 2008
(86) PCT No.: PCT/EP2008/059290

§ 371 Date: Jan. 26, 2010

(30) Foreign Application Priority Data
Jul. 26, 2007 (EP) 07113268.2

(51) Int. Cl. C07C 253/30; A61K 8/40

(52) U.S. Cl. 512/6; 558/373

(57) ABSTRACT

The present invention relates to a method of producing ethylgeranonitrile through reaction of ethylheptenone with a deprotonated nitrile and, if appropriate, subsequent saponification and decarboxylation. Moreover, the invention relates to mixtures comprising 3,7-dimethyl-2,6-nonadienenitrile and at least one compound selected from the group of compounds 3,7-dimethyl-3,6-nonadienenitrile and 3-methylene-7-methyl-6-nonenenitrile, and to fragrance compositions comprising these.

The present invention relates to a method of producing ethylgeranonitrile through reaction of ethylheptenone with a deprotonated or enolized nitrile and, if appropriate, subsequent saponification and decarboxylation. Moreover, the invention relates to mixtures comprising 3,7-dimethyl-2,6-nonadienenitrile and at least one compound selected from the group of compounds 3,7-dimethyl-3,6-nonadienenitrile and 3-methylene-7-methyl-6-nonenenitrile.

Aliphatic nitriles are valuable aroma chemicals which, particularly on account of the chemical stability of the nitrile group, are suitable for use under normeutral conditions, for example under basic or strongly acidic conditions. Consequently, there is a continuing need for novel aliphatic nitriles or mixtures thereof with interesting odor properties, and also for methods for the inexpensive production of such substances or mixtures of substances.

One of the aliphatic nitriles most in demand on account of its special fragrance properties is geranonitrile (3,7-dimethyl-2,6-octadienenitrile) which has a very intensely citrus-like odor profile with bitter and cuminic and also fresh-hay-like facets. An alternative to this is 3,7-dimethyl-2,6-nonadienenitrile or ethylgeranonitrile, also sold as Lemonile®, which in significant aspects has equivalent or even advantageous odiferous facets. This product conveys a more natural radiance with a longer lasting effect and reduced bitter facets and is accordingly very well suited as a substitute for geranonitrile.

Since geranonitrile is assessed as a so-called cmr2 substance on the basis of recent toxicological findings, it is to be taken into account that the industrial production is to cease in the future.

Methods of producing α,β-unsaturated nitriles are known to the person skilled in the art. For example, DE 21 35 666 discloses a method of producing cycloalkylidene-acetonitriles by reacting cycloalkanones with acetonitrile in liquid phase in the presence of catalytic amounts of alkali metal or alkaline earth metal alkoxides.

U.S. Pat. No. 3,960,923 relates to a method of producing α,β-unsaturated nitriles by reacting a ketone with acetonitrile in the presence of a strong base. Aliphatic ketones, including methyl heptenone, are specified as suitable ketones.

EP 0 074 253 discloses special saturated, α- or β-substituted aliphatic nitriles, their use as fragrances, and methods for their production. For example, the reaction of methyl alkyl ketones with tosylmethylisocyanide with potassium tert-butoxide in diglyme is described. In the course of a further method, methyl alkyl ketones are reacted with cyanoacetic acid in the presence of ammonium acetate.

U.S. Pat. No. 4,361,702 relates to a method of producing acetals of 3-formyl-2-butenenitrile by reacting the correspondingly acetalized methyl ketone with acetonitril in the presence of a strong base.

S. DiBiase et al. describe in Org. Synth. Coll. Vol. 7, 1990, 108-112 and Org. Synth. Vol. 62, 1984, 179-185 the reaction of cyclohexanone or benzaldehyde with acetonitrile in the presence of potassium hydroxide to give the corresponding α,β-unsaturated nitriles.

Moreover, S. DiBiase et al. describe in J. Org. Chem., Vol. 44, 1979, 4640-4649 the production of α,β-unsaturated nitriles by reacting carbonyl compounds with acetonitrile in the presence of bases and, if appropriate, crown ethers.

A. Valla et al. describe in Synthetic Communications, Vol. 33, No. 7, 2003, pp. 1195-1201, the reaction of 6-methyl-5-hepten-2-one with acetonitrile in the presence of potassium hydroxide to give geranonitrile in a yield of 65%.

U.S. Pat. No. 3,655,722 relates to 7-methyl-3-methylene-6-octenenitrile and 3,7-dimethyl-2,6-octadienenitrile (geranonitrile), and to a mixture of the cis and trans isomers of geranonitrile and further double-bond isomers of 3,7-dimethyloctadienenitrile. The compounds are obtained by reacting 2-methyl-2-hepten-6-one with cyanoacetic acid in the presence of triethanolamine or cyclohexylamine.

The object of the present invention was to provide a method of producing ethylgeranonitrile. The method should permit the preparation of ethylgeranonitrile or isomer mixtures thereof and be practicable in the simplest way possible in terms of processing on an industrial scale. Starting from inexpensive, readily available starting materials, it should produce the desired product in the highest possible yield while avoiding undesired by-products. Moreover, the product should be produced in a form, i.e. in the form of isomer mixtures, in which it is suitable as substitute for geranonitrile. In particular, it should have an odor profile that is comparable with that of geranonitrile, as far as possible of equal ranking and preferably more pleasant or more attractive, and ensure easy substitutability of the geranonitrile.

The object was achieved through the provision of a method for producing 3,7-dimethyl-2,6-nonadienenitrile comprising the steps
a) reaction of a nitrile of the formula (II)

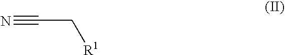

in which the radical
R¹ is hydrogen or the radical —C(O)OR², where
R² if appropriate is hydrogen or straight-chain, branched or cyclic C₁-C₆-alkyl,
with an activation reagent to give the nitrile of the formula (II) in deprotonated or enolized form and
b) reaction of the nitrile of the formula (II) in deprotonated or enolized form with 6-methyl-5-nonen-2-one to give 3,7-dimethyl-2,6-nonadienenitrile or of a compound of the formula (IV)

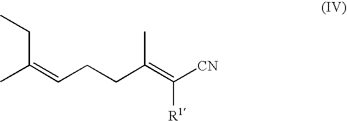

in the form of an isomer mixture with regard to the ethylenic double bonds,
where the radical
R^{1′} is —C(O)OR² and
R² is hydrogen or straight-chain, branched or cyclic C₁-C₆-alkyl,
and in the case where an isomer mixture of the compound of the formula (IV) is obtained,
c) if appropriate saponification of the resulting compound of the formula (IV) to give the corresponding free carboxylic acid and subsequent decarboxylation to give 3,7-dimethyl-2,6-nonadienenitrile.

The method according to the invention is suitable for producing 3,7-dimethyl-2,6-nonadienenitrile of the formula (I)

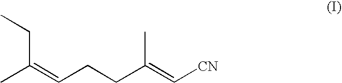

where the compound is usually produced in the form of a mixture of configuration isomers (i.e. E/Z isomers) with regard to the two ethylenic double bonds and in the form of mixtures with constitution isomers with regard to the double bond in α,β or β,γ position relative to the nitrile group.

Possible configuration isomers, which are usually obtained besides the isomer of the formula (I), are the compounds (Ia) to (Ic)

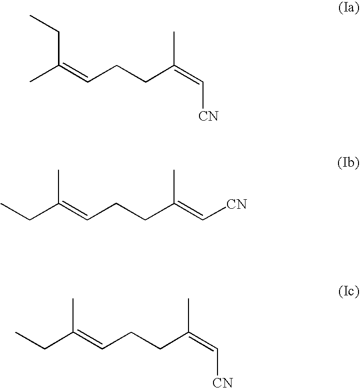

which differ from one another merely as regards the E/Z configuration of the ethylenic double bonds. 3,7-dimethyl-2,6-nonadienenitrile, i.e. the compounds of the formulae (I) and (Ia) to (Ic) has or have an ethylenic double bond in α,β position relative to the nitrile group and, within the scope of the present invention, are also referred to as conjugated isomers.

Possible constitution isomers, which can be obtained besides the 3,7-dimethyl-2,6-nonadienenitrile usually obtained as main product, are the compounds 3,7-dimethyl-3,6-nonadienenitrile and 3-methylene-7-methyl-6-nonenenitrile. 3,7-dimethyl-3,6-nonadienenitrile and 3-methylene-7-methyl-6-nonenenitrile can likewise be obtained in the form of mixtures of the configuration isomers, i.e. E/Z isomers. Thus, 3-methylene-7-methyl-6-nonenenitrile is usually obtained in the form of a mixture of the two isomers of the formulae (V) and (Va)

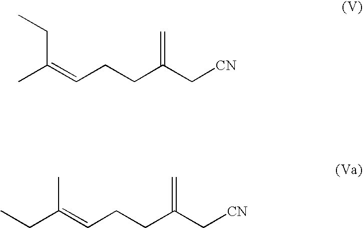

3,7-dimethyl-3,6-nonadienenitrile is usually obtained in the form of mixtures of all four configuration isomers of the formulae (VI) and (VIa) to (VIc)

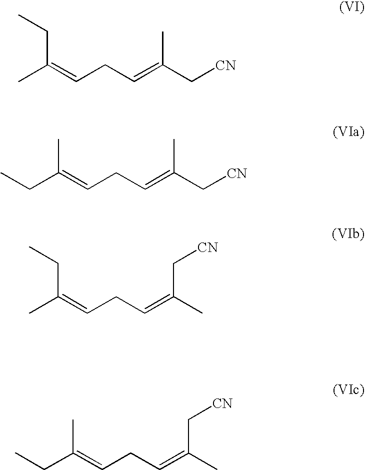

3,7-dimethyl-3,6-nonadienenitrile and 3-methylene-7-methyl-6-nonenenitrile have an ethylenic double bond in β,γ position relative to the nitrile group and, within the scope of the present invention, are together also referred to as nonconjugated isomers.

The mixtures accessible according to the invention usually comprise 3,7-dimethyl-2,6-nonadienenitrile (i.e. the compound of the formula (I) and its isomers of the formula (Ia) to (Ic)) as main component in a fraction of generally about 40 to about 100% by weight, preferably 50 to 100% by weight, and particularly preferably 60 to 100% by weight, based on the resulting isomer mixture of the compounds of the formulae (I), (V) and (VI) and isomers thereof.

The specified nonconjugated isomers, i.e. 3,7-dimethyl-3,6-nonadienenitrile and 3-methylene-7-methyl-6-nonenenitrile, are each usually formed, depending on the choice of reaction conditions, to a lesser degree, i.e. in total in a degree of up to about 60% by weight, preferably up to 50% by weight and particularly preferably up to 40% by weight, based on the resulting mixture, or are accordingly produced only to a slight degree or not at all.

Serving as starting material for carrying out the method according to the invention are nitriles of the formula (II)

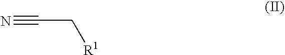

in which the radical R¹ is hydrogen or the radical —C(O)OR², where the radical R² can be hydrogen or straight-chain, branched or cyclic, preferably straight-chain or branched, C₁-C₆-alkyl. If the radical R¹ is hydrogen, acetonitrile accordingly serves as starting material of the formula (II). For cases where R¹ is the radical —C(O)OR², cyanoacetic acid or esters thereof serve as starting materials of the formula (II), where the specified esters can be straight-chain, branched or cyclic, preferably straight-chain or branched, C₁-C₆-alkyl radicals, such as, for example, methyl, ethyl, propyl, isopropyl, n-butyl, sec-butyl, tert-butyl, n-pentyl, isopentyl, neopentyl, cyclopentyl, n-hexyl or cyclohexyl, preferably methyl, ethyl, isopropyl, particularly preferably methyl and ethyl.

Within the scope of a preferred embodiment of the method according to the invention, the radical R¹ is hydrogen. A nitrile of the formula (II) preferred according to the invention is accordingly acetonitrile. Further nitriles of the formula (II) preferred according to the invention are cyanoacetic acid, methyl cyanoacetate and ethyl cyanoacetate, which can be represented in summary by formula (IIa), where the radical R² can have the abovementioned meanings.

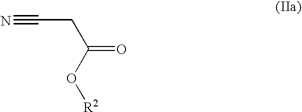

The selected nitrile of the formula (II) is reacted according to step a) of the method according to the invention with an activation reagent to give the nitrile of the formula (II) in deprotonated or enolized form. The term activation reagent is to be understood here as meaning those reagents which can deprotonate, or if using cyanoacetic acid or cyanoacetic esters of the formula (IIa) enolize, the nitrile used, preferably acetonitrile, cyanoacetic acid or cyanoacetic esters, preferably methyl or ethyl esters, in a position, i.e. on the carbon atom adjacent to the nitrile group, preferably those reagents which have a pKa value of 4 or above, i.e. weak acids or bases, preferably bases (Brönstedt bases). This gives the nitrile of the formula (II) in deprotonated or enolized form, as represented, for example, by formula (IIb), where the activation reagent used was a base and M⁺ is the counterion of the base used:

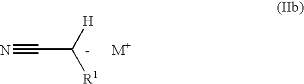

In the case of the starting materials of the formula (IIa) according to the invention, i.e. when using cyanoacetic acid or cyanoacetic esters, the same are obtained as intermediates at least to a slight degree in enolized form, i.e. in the form of their enols or enolates of the formula (IIc), where M⁺ is a proton (H⁺) or the counterion of the selected base:

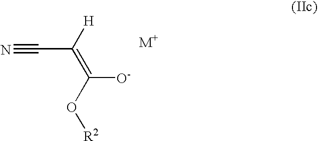

In the case of the use according to the invention of alkali metal hydroxides, such as sodium hydroxide (NaOH) or potassium hydroxide (KOH) as activation reagents or bases, the counterions M⁺ in the aforementioned formulae (IIa) and (IIb) would accordingly be understood as meaning Na⁺ or K⁺.

Within the scope of a preferred embodiment of the method according to the invention, the activation reagent used is a base or a mixture of different bases. Suitable bases in this regard which may be mentioned are those which have a pKa value of from about 5 to about 50, preferably about 9 to about 25, where the term pKa value is as defined in Organikum 21st edition, Weinheim, Wiley-VCH, 2001, pp. 156-159. Examples of bases to be used with preference according to the invention are; alkali metal or alkaline earth metal hydroxides, such as, for example; LION, NaOH, KOH, Ba(OH)₂; ammonium hydroxides; alkali metal or alkaline earth metal alkoxides of short-chain alcohols, preferably those having 1 to 6, preferably 1 to 4, carbon atoms, such as, for example, sodium methoxide, sodium ethoxide, potassium methoxide, potassium ethoxide, sodium propoxide, potassium propoxide, sodium isopropoxide, potassium isopropoxide, sodium butoxide, potassium butoxide, sodium isobutoxide, potassium isobutoxide.

Further activation reagents that can be used according to the invention for the reaction of cyanoacetic acid or cyanoacetic esters are primary, secondary and tertiary amines, and amino acids and ammonium salts, such as, for example; piperidine, pyridine, imidazole and derivatives thereof, diazabicycloundecane (DBU), diazabicyclononane (DBN), 1,4-diazabicyclo[2.2.2]octane (DABCO), N,N-dimethylaminopyridine (DMAP), quinoline, p-aminophenol, β-alanine, lysine, ammonium acetate. These can be used individually or in the form of mixtures. If appropriate, it may be advantageous to additionally carry out the reaction in the presence of an acid, such as, for example, glacial acetic acid. Furthermore, organometallic compounds, such as, for example, butyllithium or amides, such as, for example, lithium amide or sodium amide, can generally be used for the reaction of acetonitrile.

When using the nitrile preferred according to the invention, acetonitrile, the activation reagent used is advantageously bases with a pKa value of from 12 to 50, preferably with a pKa value of from 15 to 45.

The selected activation reagent can be used stoichiometrically or catalytically in a ratio of 0.01 to 10 mol/mol, preferably 0.1 to 5 mol/mol, particularly preferably 0.5 to 1.5 mol/mol of carbonyl compound (6-methyl-5-octen-2-one).

In addition, it may be advantageous, depending on the type of activation reagent used, to add additives, such as, for example, crown ethers, phase transfer catalysts or acids, such as, for example, glacial acetic acid, to the reaction mixture.

The specified activation reagents can be used as such in solid form, if appropriate, liquid form or in the form of solutions in suitable solvents or mixtures thereof. The reaction according to step a) of the method according to the invention is usually undertaken in such a way that the selected nitrile of the formula (II) is initially introduced as such or in the form of a solution in a suitable solvent inert under the reaction conditions, such as, for example, benzene, toluene, xylene, pentane, hexane, cyclohexane, heptane, diethyl ether, petroleum ether, 1,4-dioxane, tetrahydrofuran, N,N-dimethylformamide (DMF), methanol, ethanol, propanol, butanol, methylene chloride or chloroform, and the selected activation reagent, i.e. preferably the selected base, is added. In one embodiment preferred according to the invention, the reaction is carried out in acetonitrile as solvent. This can then also serve as starting material of the formula (II). A further advantage of acetonitrile as solvent is its ability, by forming acetamide, to remove water which forms during the reaction from the mixture. Other scavengers, such as, for example, molecular sieve, can likewise be added.

According to step b) of the method according to the invention, the nitrile of the formula (II) obtained as described above is reacted in deprotonated or enolized form with 6-methyl-5-octen-2-one, which, within the scope of the present invention, is also referred to as ethylheptenone and is used in the form of a mixture of the E/Z isomers of the formula (III) and (IIIa)

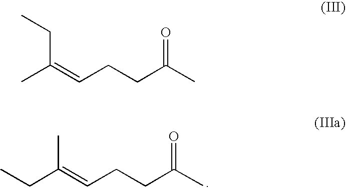

The reaction steps a) and b) according to the invention can be carried out separately one after the other or in a reaction mixture of the selected nitrile of the formula (II), the selected activation reagent and the ethylheptenone. The latter variant has proven useful in particular for reactions with cyanoacetic acid with buffer systems such as, for example, ammonium acetate or piperidine, β-alanine in the presence of glacial acetic acid.

The nitriles of the formula (II) to be used as starting materials within the scope of the method according to the invention, in particular cyanoacetic acid and esters thereof of the formula (IIa), and also ethylheptenone are usually reacted together in an approximately equimolar ratio, where, in particular, the more conveniently available cyanoacetic acid or esters thereof can be used in slight excess, generally in a molar ratio relative to the ethylheptenone used of, for example, 1.05:1 to about 2:1, preferably about 1.1:1 to about 1.5:1. One exception here is acetonitrile, which can also be used as solvent in large excess.

The amount of base added can be varied over a wide range since the reaction can be carried out in catalytic or stoichiometric form depending on the strength of the base used and also depending on the choice of starting material of the formula (II).

Within the scope of a preferred embodiment, the method according to the invention involves firstly, in the course of step a) according to the invention, deprotonating the nitrile of the formula II used completely or predominantly to give the corresponding anion by reaction with an approximately equimolar amount, usually an amount of from about 0.9 to about 1.2 mol equivalents, preferably about 0.95 to about 1.1 mol equivalents, of a suitable base as described above, and then reacting it according to step b) with ethylheptenone. Within the scope of a further preferred embodiment, the method according to the invention is carried out by initially introducing the reaction mixture obtained according to step a), and metering in ethylheptenone, preferably slowly.

Within the scope of a preferred embodiment of the method according to the invention, acetonitrile is used according to step a); this is converted with a strong base selected from the group of bases comprising the alkali metal or alkaline earth metal hydroxides, ammonium hydroxide and alkali metal or alkaline earth metal alkoxides, in an amount of from about 0.95 to 1.2 mol equivalents (based on the ethylheptenone to be used in step b)) initially completely or for the most part into the corresponding deprotonated acetonitrile. Here, the acetonitrile can also be used as solvent in (large) excess. The reaction usually proceeds rapidly, depending on the selected base and the reaction temperature, and, when using an alkali metal alkoxide such as, for example, sodium methoxide, at room temperature, when using an alkali metal hydroxide such as, for example, KOH, at, if appropriate, slightly elevated temperatures of up to about 80° C., is usually largely completed within a few minutes to about 1 h.

The thus prepared solution of the deprotonated nitrile can then be reacted according to step b) with the ethylheptenone. This advantageously takes place with slow metered addition of the ketone in order to avoid undesired secondary reactions as far as possible.

The reaction of the ethylheptenone with the deprotonated or enolized nitrile of the formula (II) likewise takes place rapidly and is usually largely complete after about 1 to about 24 h at temperatures of from about 20 to about 180° C. Depending on the type of reaction procedure and depending on the solvent used, it may be advantageous to carry out the reaction according to the invention with ethylheptenone with elimination of the water that forms during the reaction in stoichiometric amount. For this purpose, it may in some cases also be advantageous to add an additional inert solvent, such as, for example, toluene or xylene, which serves as entrainer for the resulting water of reaction.

The reaction of the deprotonated nitrile of the formula (II) with ethylheptenone leads, in the case of the reaction of acetonitrile, in a manner according to the invention directly to the 3,7-dimethyl-2,6-nonadienenitrile or to the isomer mixture comprising 3,7-dimethyl-2,6-nonadienenitrile. Carrying out the method using cyanoacetic acid or esters thereof of the formula (IIa), i.e. in cases where the radical R¹ in formula (II) is not hydrogen, produces, according to step b), firstly the intermediates of the formula (IV)

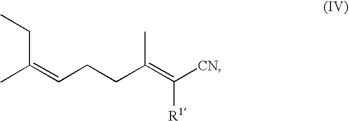

in which the radical R^{1′} is the hydroxy- or alkoxycarbonyl group-C(O)OR² and R² is hydrogen or straight-chain, branched or cyclic, preferably straight-chain or branched, C₁- to C₆-alkyl as described above, where the radial R^{1′} is assigned the same meaning as the radical R¹ of the compound of the formula (II) or (IIa) used in each case. The compound of the formula (IV) is here usually likewise obtained in the form of a mixture with the other E/Z double-bond isomers.

In cases where the radical R¹ of the nitrile of the formula (II) used is not hydrogen, firstly according to step b) of the method according to the invention, the esters or the carboxylic acids of the formula (IV) and also their configuration isomers with regard to the ethylenic double bonds are accordingly firstly obtained at least as intermediates and, according to an additional step c) if appropriate saponified to give the free carboxylic acid and then decarboxylated to give 3,7-dimethyl-2,6-nonadienenitrile or isomer mixtures comprising this. In this connection, with suitable selection of the reaction conditions, specifically when using stoichiometric amounts of base, the specified saponification and decarboxylation can take place in situ within the course of the reaction according to step b) of the method according to the invention. In another case, the specified saponification and decarboxylation can be carried out by methods known to the person skilled in the art.

The reaction mixtures obtained according to the invention can be further worked-up by methods known to the person skilled in the art, for example by means of extractive methods and then further purified, for example by distillative methods.

Within the scope of a further preferred embodiment, the product mixtures obtained according to the invention, in particular the product mixtures or crude product mixtures obtained in the reaction of the compounds of the formula (IIa), i.e. when using cyanoacetic acid or cyanoacetic esters, by the method according to the invention, are subjected to a downstream isomerization by treatment or contacting with a base, preferably a strong base. Bases suitable for the purposes of the downstream additional isomerization are the alkali metal alkoxides and hydroxides, and also the alkaline earth metal alkoxides, such as, for example, sodium methoxide, sodium ethoxide, sodium propoxide, sodium butoxide, potassium methoxide, potassium ethoxide and the like. These can be used in catalytic or stoichiometric amounts or in excess and usually in amounts of from 0.01 mol to 10 mol/mole, preferably 0.1 to 1 mol/mole, of the isomer mixture. Examples of suitable solvents which should be inert toward the starting materials or reagents used under the reaction conditions are: benzene, toluene, xylene, methanol, ethanol, propanol, butanol. Suitable reaction temperatures for carrying out the after isomerization are temperatures in the range from −80° C. to 120° C., preferably from 0 to 50° C. The isomerizations usually proceed rapidly and are usually at least largely complete after reaction times of from about 1 to about 48 h, often after about 1 to about 5 h.

The method according to the invention permits the production of 3,7-dimethyl-2,6-nonadienenitrile (ethylgeranonitrile), which is usually produced in the form of mixtures of the configuration isomers of the formula (I), (Ia), (Ib) and (Ic). Besides the specified conjugated isomers, the mixtures accessible according to the invention usually also comprise the nonconjugated, constitution-isomeric compounds 3,7-dimethyl-3,6-nonadienenitrile and 3-methylene-7-methyl-6-nonenenitrile of the formulae (V) and (Va) and/or (VI), (VIa), (VIb) and (VIc). The present invention therefore also relates to the mixtures comprising the 3,7-dimethyl-2,6-nonadienenitrile obtainable by the method according to the invention.

The method according to the invention opens up the possibility, particularly when carrying out the process variant described as preferred embodiment using acetonitrile as starting material of the formula (II) for the reaction with ethylheptenone, or when using cyanoacetic acid or esters thereof of the formula (IIa), particularly in the case of downstream isomerization as described above, of producing mixtures or raw product mixtures with a particularly low content of the specified nonconjugated isomers 3,7-dimethyl-3,6-nonadienenitrile and/or 3-methylene-7-methyl-6-nonenenitrile.

In a further aspect, the invention therefore relates to mixtures comprising 3,7-dimethyl-2,6-nonadienenitrile and at least one compound selected from the group of the compounds 3,7-dimethyl-3,6-nonadienenitrile and 3-methylene-7-methyl-6-nonene-nitrile, where the content of 3,7-dimethyl-3,6-nonadienenitrile and 3-methylene-7-methyl-6-nonenenitrile together, based on the amount of 3,7-dimethyl-2,6-nonadiene-nitrile in the mixture, is up to 10% by weight. Here and below, the specified compounds in each case comprise the possible E/Z double-bond isomers with regard to the ethylenic double bonds.

Preferably, the content of 3,7-dimethyl-3,6-nonadienenitrile and 3-methylene-7-methyl-6-nonenenitrile in the mixtures according to the invention is together 0.01 to 10% by weight, particularly preferably 0.05 to 9% by weight, preferably up to 8% by weight, in turn preferably up to 7% by weight and yet more preferably up to 6% by weight. Particularly preferably, the combined content of 3,7-dimethyl-3,6-nonadienenitrile and 3-methylene-7-methyl-6-nonenenitrile is 0.75 to 5% by weight, preferably 1 to 5% by weight, in each case based on the amount of 3,7-dimethyl-2,6-nonadienenitrile in the mixture.

The mixtures according to the invention comprise the specified compounds 3,7-dimethyl-2,6-nonadienenitrile, 3,7-dimethyl-3,6-nonadienenitrile and/or 3-methylene-7-methyl-6-nonenenitrile in a combined fraction (based on the total mixture) of from usually 80 to 100% by weight, preferably 85 or 90 to 99.9% by weight, particularly preferably 95 to 99.5% by weight, especially preferably up to 99 or up to 98% by weight. Mixtures preferred according to the invention consist essentially, i.e. to at least 90% by weight, preferably to at least 95% by weight, particularly preferably to at least 98% by weight, and very particularly preferably to at least 99% by weight, of the compounds 3,7-dimethyl-2,6-nonadienenitrile, 3,7-dimethyl-3,6-nonadienenitrile and/or 3-methylene-7-methyl-6-nonenenitrile and to a small degree, preferably in an amount of from 0.1 to 2% by weight, preferably from 0.5 to 1% by weight, of further components and/or impurities.

Mixtures particularly preferred according to the invention are those as described above which consist essentially of the compounds 3,7-dimethyl-2,6-nonadienenitrile and 3,7-dimethyl-3,6-nonadienenitrile, where the fraction of 3,7-dimethyl-3,6-nonadienenitrile is up to 10% by weight, preferably 0.01 to 10% by weight, particularly preferably 0.05 to 9% by weight, preferably up to 8% by weight, again preferably up to 7% by weight and yet more preferably up to 6% by weight, based on the amount of 3,7-dimethyl-2,6-nonadienenitrile in the mixture. The content of 3,7-dimethyl-3,6-nonadienenitrile in these mixtures is especially preferably 0.75% by weight to 5% by weight, preferably 1% by weight to 5% by weight.

Accordingly, the present invention relates to 3,7-dimethyl-2,6-nonadienenitrile (ethylgeranonitrile), which comprises up to 10% by weight, preferably up to 8% by weight, again preferably up to 7% by weight and yet more preferably up to 6% by weight, of 3,7-dimethyl-3,6-nonadienenitrile and/or 3-methylene-7-methyl-6-nonenenitrile, based on the sum of the two compounds and the total weight of the mixture). Particularly preferably, the content of 3,7-dimethyl-3,6-nonadienenitrile and/or 3-methylene-7-methyl-6-nonenenitrile in the 3,7-dimethyl-2,6-nonadienenitrile is 0.75% by weight to 5% by weight, preferably 1% by weight to 5% by weight.

The mixtures according to the invention have an intense lemon-like scent with natural radiance. The mixtures according to the invention are advantageous substitutes for geranonitrile (3,7-dimethyl-2,6-octadienenitrile), particularly in cases where mixtures have a high purity with regard to the (E,Z)-2,6-configured double-bond isomers, i.e. with regard to the conjugated isomers. The mixtures according to the invention are therefore suitable to a particular degree as alternatives or replacement for the fragrance geranonitrile.

The present invention therefore also relates to the use of the mixtures described above, in particular of the mixtures comprising 3,7-dimethyl-2,6-nonadienenitrile and at least one compound selected from the group of the compounds 3,7-dimethyl-3,6-nonadienenitrile and 3-methylene-7-methyl-6-nonenenitrile, where the content of 3,7-dimethyl-3,6-nonadienenitrile and 3-methylene-7-methyl-6-nonenenitrile together, based on the amount of 3,7-dimethyl-2,6-nonadienenitrile in the mixture, is up to 10% by weight, as fragrances.

In particular, the mixtures according to the invention are suitable as fragrances for producing a citrus odor, as characterized above. Accordingly, in a further aspect, the present invention relates to the use of the mixtures according to the invention as fragrances for producing a lemon scent.

The mixtures accessible according to the invention as described above can, for the aromatization thereof, be incorporated into consumer articles or everyday commodities or applied to such and thereby impart to them a pleasant lemon-like scent. Examples of consumer articles or everyday commodities that can be aromatized with the mixtures according to the invention are: cleaning compositions, such as, for example, scouring compositions, cleaners, care compositions for the treatment of surfaces, for example of furniture, floors, kitchen appliances, glass panes and windows, and also windscreens, detergents, softeners, laundry treatment compositions, textile treatment compositions, such as, for example, ironing aids, and also bleaches and bleach liquors, toilet blocks, limescale removers, air fresheners (air care), fragrance compositions, for example for fine perfumery, cosmetic compositions, but also fertilizers, building materials, mold removers, disinfectants, products for car care and the like.

On account of their chemical similarity to the fragrance geranonitrile, the mixtures according to the invention are suitable in particular for the customary applications of geranonitrile which, on account of its chemical stability, can advantageously also be used in aggressive media, such as acids or alkalis or bleaching liquors, for example in toilet cleaners or blocks and, for example, also in effluent or tube cleaners, and, for example, also in grill or oven cleaners or in other metal cleaners.

In a further aspect, the present invention therefore also relates to consumer articles or everyday commodities or compositions comprising an organoleptically effective amount of the mixtures according to the invention comprising 3,7-dimethyl-2,6-nonadienenitrile and at least one compound selected from the group of the compounds 3,7-dimethyl-3,6-nonadienenitrile and 3-methylene-7-methyl-6-nonenenitrile, where the content of 3,7-dimethyl-3,6-nonadienenitrile and 3-methylene-7-methyl-6-nonenenitrile together, based on the amount of 3,7-dimethyl-2,6-nonadienenitrile in the mixture, is up to 10% by weight.

Moreover, it has been found that the ethylgeranonitrile (3,7-dimethyl-2,6-nonadienenitrile) accessible according to the invention and, in particular, the mixtures according to the invention comprising 3,7-dimethyl-2,6-nonadienenitrile and at least one compound selected from the group of the compounds 3,7-dimethyl-3,6-nonadiene-nitrile and 3-methylene-7-methyl-6-nonenenitrile, where the content of 3,7-dimethyl-3,6-nonadienenitrile and 3-methylene-7-methyl-6-nonenenitrile together, based on the amount of 3,7-dimethyl-2,6-nonadienenitrile in the mixture, is up to 10% by weight, are suitable to a particular degree for mixing with the fragrances citronellylnitrile (3,7-dimethyl-6-octenenitrile) of the formula (VII)

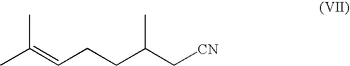

and/or dihydrocitronellylnitrile (3,7-dimethyloctanenitrile) of the formula (VIII),

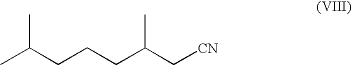

where in general any desired mixing ratios can be used.

Therefore, in a further aspect, the present invention relates to fragrance compositions comprising 3,7-dimethyl-2,6-nonadienenitrile and at least one further component selected from the fragrances dihydrocitronellylnitrile (3,7-dimethyloctanenitrile) and citronellylnitrile (3,7-dimethyl-6-octenenitrile). Fragrance compositions of this type are particularly suitable for the substitution of the fragrance geranonitrile. Accordingly, the fragrance compositions according to the invention comprise ethylgeranonitrile (3,7-dimethyl-2,6-nonadienenitrile) and, if desired, citronellylnitrile or dihydrocitronellylnitrile, preferably citronellylnitrile and dihydrocitronellylnitrile or particularly preferably only citronellylnitrile. In this connection, within the scope of a preferred embodiment, ethylgeranonitrile can also be used in the form of the mixture, accessible according to the invention, of 3,7-dimethyl-2,6-nonadienenitrile and at least one compound selected from the group of the compounds 3,7-dimethyl-3,6-nonadienenitrile and 3-methylene-7-methyl-6-nonenenitrile, where the content of 3,7-dimethyl-3,6-nonadienenitrile and 3-methylene-7-methyl-6-nonenenitrile together, based on the amount of 3,7-dimethyl-2,6-nonadienenitrile in the mixture, is up to 10% by weight.

Within the scope of this aspect of the present invention, particularly preferred fragrance compositions are those which, in each case based on the total weight of the finished fragrance composition, comprise 1 or preferably 2 to 20% by weight, preferably 5 to 10% by weight, of dihydrocitronellylnitrile, 20 to 60% by weight, preferably 35 to 45% by weight, of citronellylnitrile and 35 to 75% by weight, preferably 50 to 60% by weight, of 3,7-dimethyl-2,6-nonadienenitrile or a mixture of 3,7-dimethyl-2,6-nonadienenitrile and at least one compound selected from the group of the compounds 3,7-dimethyl-3,6-nonadienenitrile and 3-methylene-7-methyl-6-nonenenitrile, where the content of 3,7-dimethyl-3,6-nonadienenitrile and 3-methylene-7-methyl-6-nonenenitrile together, based on the amount of 3,7-dimethyl-2,6-nonadienenitrile in the mixture, is up to 10% by weight. Such fragrance compositions have, as far as fine nuances, analogies to the scent impression of geranonitrile and can be used in a similar way and in approximately equal concentration to the geranonitrile to be substituted.

The fragrance compositions according to the invention can be diluted as desired with the solvents customary in this field of application. Examples of suitable solvents are: ethanol, dipropylene glycol or ethers thereof, phthalates, propylene glycols, or carbonates of diols, preferably ethanol. Water is also suitable as solvent for diluting the fragrance compositions according to the invention and can advantageously be used together with suitable emulsifiers.

A preferred embodiment of the fragrance composition according to the invention is characterized in that it comprises ethylgeranonitrile in the form of a mixture comprising 3,7-dimethyl-2,6-nonadienenitrile and at least one compound selected from the group of the compounds 3,7-dimethyl-3,6-nonadienenitrile and 3-methylene-7-methyl-6-nonenenitrile, where the content of 3,7-dimethyl-3,6-nonadienenitrile and 3-methylene-7-methyl-6-nonenenitrile together, based on the amount of 3,7-dimethyl-2,6-nonadiene-nitrile in the mixture, is up to 10% by weight.

Such mixtures and their preferred embodiments as described above within the scope of the present invention are suitable to a particular degree for incorporation into the fragrance compositions according to the invention.

On account of the structural and chemical similarity of the components, the fragrance compositions according to the invention have high stability and durability, are characterized by a fresh lemon-like odor and thus likewise represent suitable substitutes for the fragrance geranonitrile. In this connection, it is to be emphasized as advantageous that the two possible components of the fragrance composition citronellylnitrile and dihydrocitronellylnitrile in each case represent inexpensive compounds that are readily accessible on an industrial scale and therefore contribute to being able to reduce the requirement for ethylgeranonitrile accessible according to the invention or mixtures according to the invention. In a further aspect, the present invention therefore relates to the use of the fragrance compositions according to the invention for producing a citrus odor.

Like the mixtures specified above, the fragrance compositions according to the invention are likewise suitable for incorporation into consumer articles and everyday commodities or compositions as have been described by way of example above for the specified mixtures. A further aspect of the present invention therefore relates to consumer articles or everyday commodities which comprise an organoleptically effective amount of the fragrance compositions according to the invention, where the fragrance composition can be incorporated into the specified articles, or else be applied thereto. Here, for the purposes of the overall present invention, an organoleptically effective amount is to be understood as meaning an amount which suffices, when used as intended, to bring about a scent impression for the user or consumer, specifically the impression of an intense citrus odor.

The examples below serve to illustrate the invention without limiting it in any way:

### EXAMPLE 1

84.8 g (1.0 mol) of cyanoacetic acid, 280 g (2.0 mol) of ethylheptenone and 192 g (2.4 mol) of pyridine were initially introduced into 360 ml of toluene. 6.6 g (0.08 mol) of ammonium acetate were added and the mixture was heated under reflux in the water separator. After a reaction time of 1 h, a further 42.4 g (0.5 mol) of cyanoacetic acid were added per hour over a period of 7 h and the reaction mixture was afterstirred while removing water azeotropically. After a run time of 12 h, about 250 ml of solvent were distilled off and the mixture was stirred for a further 3 h at reflux temperature.

The organic phase was then washed twice with 200 ml of saturated NaHCO₃ solution and the solvent was removed in vacuo. This gave 297 g of a crude product, which was analyzed by gas chromatography.

GC-method:

Column: 50 m CP-SIL 13 CB

ID: 0.32 mm FD: 1.2 μm 90° C.; 2° C./min to 200° C.; 6° C./min; 290° C., 20 min isotherm.

The crude product had the composition given in table 1 (all data in GC area %), where the content of the unassigned configuration (E/Z) isomers possible in each case, with regard to the respective ethylenic double bond(s) has been given.

TABLE 1

| Product (GC area %) | 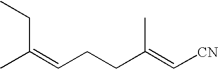 | | | | 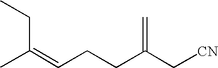 | | 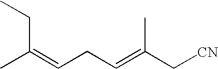 | | | |
|---|---|---|---|---|---|---|---|---|---|---|
| 89.4% | 20.1% | 9.8% | 0% | 19.0% | 13.5% | 5.9% | nd | 4.1% | 27.6% | 0% |

nd = not determined

The crude product obtained in this way was subjected to fractional distillation at a bottom temperature of from 106 to 135° C. and an absolute pressure of 3 mbar. This gave 210.2 g (1.29 mol corresponding to 64.5% of theory) of ethylgeranonitrile in the form of a mixture of double-bond isomers.

### EXAMPLE 2

84.8 g (1.0 mol) of cyanoacetic acid, 280 g (2.0 mol) of ethylheptenone and 192 g (2.4 mol) of pyridine were initially introduced into 360 ml of toluene. 6.6 g (0.08 mol) of ammonium acetate were added and the mixture was heated under reflux in a water separator. After a reaction time of 1 h, a further 42.4 g (0.5 mol) of cyanoacetic acid were added per hour over a period of 7 h and the reaction mixture was afterstirred while removing water azeotropically. After a runtime of 12 h, about 250 ml of solvent were distilled off and the mixture was stirred for a further 3 h at reflux temperature.

After this time had passed, the organic phase was washed twice with 200 ml of saturated NaHCO₃ solution. Then, 180 g (1.0 mol) of sodium methoxide (30% strength solution in methanol) were added dropwise to the crude solution at room temperature. The reaction mixture was stirred for 1 h at room temperature and then neutralized by adding 100 ml of acetic acid (50% strength solution in water). The solvent was distilled off in vacuo. This gave 317 g of a crude product, which was analyzed by gas chromatography.

The crude product had the composition given in table 2 (all data in GC area %), where the content of the unassigned configuration (E/Z) isomers possible in each case, with regard to the respective ethylenic double bond(s), has been given.

TABLE 2

| Product (GC area %) | 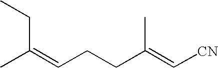 | | | | 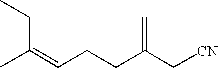 | | 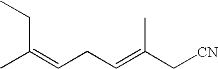 | | | |
|---|---|---|---|---|---|---|---|---|---|---|
| 87.5% | 15.5% | 23.6% | 24.2% | 35.6% | 0.1% | 0.2% | nd | 0.2% | 0.2% | 0.4% |

The crude product obtained in this way was subjected to fractional distillation at a bottom temperature of from 112 to 129° C. and an absolute pressure of from 3 to 4 mbar. This gave 216 g (1.3 mol corresponding to 65% of theory) of ethylgeranonitrile in the form of a mixture of double-bond isomers.

### EXAMPLE 3

148.5 g (1.5 mol) of methyl cyanoacetate were initially introduced into 750 ml of methanol. 270 g (1.5 mol) of sodium methoxide (30% strength solution in methanol) were added and, over the course of one hour, 210.3 g (1.5 mol) of ethylheptenone in 150 ml of methanol were added dropwise to the mixture. The reaction mixture was then heated to reflux for 22 h.

After this time had passed, the mixture was neutralized with 90 g (1.5 mol) of acetic acid and the solvent was drawn off in vacuo. The residue was admixed with 500 ml of water and 500 ml of toluene and the phases were separated. The aqueous phase was extracted with 3×100 ml of toluene. The combined organic phases were washed with 3×100 ml of water. The solvent was drawn off in vacuo. This gave 247 g of a crude product, which was analyzed by gas chromatography.

The crude product has the composition given in table 3 (all data in GC area %), where the content of the unassigned configuration (E/Z) isomers possible in each case, with regard to the respective ethylenic double bond(s), has been stated.

TABLE 3

| Product (GC area %) | 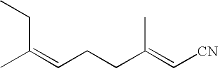 | | | | 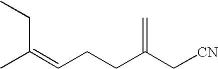 | | 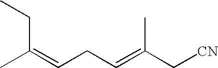 | | | |
|---|---|---|---|---|---|---|---|---|---|---|
| 62.3% | 7.1% | 14.8% | 12.6% | 26.4% | 0% | 13.1% | 1.2% | 3.5% | 6.7% | 14.5% |

The crude product obtained in this way was subjected to fractional distillation at a bottom temperature of from 135 to 210° C. and an absolute pressure of from 3 to 5 mbar. This gave 148.4 g (0.91 mol corresponding to 60.6% of theory) of ethylgeranonitrile in the form of a mixture of double-bond isomers.

### EXAMPLE 4

180 g (1 mol) of sodium methoxide (30% strength solution in methanol) were initially introduced into 410 g (10 mol) of acetonitrile and the mixture was heated to 80° C. Over the course of 1 h, 140 g (1 mol) of ethylheptenone in 200 ml of acetonitrile were added dropwise and the reaction mixture was heated under reflux overnight. 60 g (1 mol) of acetic acid were then added dropwise and the mixture was admixed with 200 ml of water. The phases were separated and the aqueous phase was extracted with 3×100 ml of toluene. The organic phases were combined and the solvent was removed in vacuo. This gave 159 g of a crude product, which was analyzed by gas chromatography.

The crude product had the composition given in table 4 (all data in GC area %), where the content of the unassigned configuration (E/Z) isomers possible in each case, with regard to the respective ethylenic double bond(s), has been stated.

TABLE 4

| Product (GC area %) | 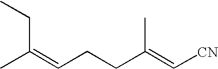 | | | | 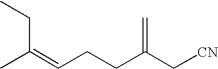 | | 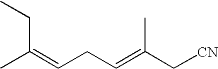 | | | |
|---|---|---|---|---|---|---|---|---|---|---|
| 80.6% | 12.4% | 26.8% | 19.1% | 40.7% | 0% | 0% | nd | 0.2% | 0.2% | 0.5% |

The crude product obtained in this way was subjected to fractional distillation at a bottom temperature of from 120 to 190° C. and an absolute pressure of from 3 to 5 mbar. This gave 92 g (0.56 mol corresponding to 56% of theory) of ethylgeranonitrile in the form of a mixture of double-bond isomers.

### EXAMPLE 5

224 g (4 mol) of KOH pellets were initially introduced into 580 g (14 mol) of acetonitrile and the mixture was heated to 80° C. Over the course of 2 h, 561 g (4 mol) of ethylheptenone were added dropwise and the reaction mixture was heated under reflux for 4 h. The reaction mixture was cooled to room temperature and then admixed with 600 ml of water and 300 ml of toluene. The phases were separated and the organic phase was washed with 3×500 ml of 25% strength sulfuric acid solution. The organic phase was dried over sodium sulfate and the solvent was removed in vacuo. This gave 674 g of a crude product, which was analyzed by gas chromatography.

The crude product had the composition given in Table 5 (all data in GC area %), where the content of the unassigned configuration (E/Z) isomers possible in each case, with regard to the respective ethylenic double bond(s), has been stated.

TABLE 5

| Product (GC area %) | 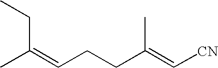 | | | | 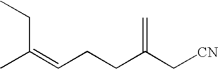 | | 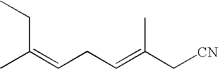 | | | |
|---|---|---|---|---|---|---|---|---|---|---|
| 68.2% | 14.4% | 23.5% | 23.0% | 36.8% | 0% | 0% | nd | 0.1% | 0.2% | 0.3% |

The crude product obtained in this way was firstly freed from high-boiling components by means of Sambay evaporator at a temperature of from 140 to 150° C. and an absolute pressure of 5 mbar and then subjected to fractional distillation at a bottom temperature of from 120 to 160° C. and an absolute pressure of 10 mbar. This gave 352 g (2.16 mol corresponding to 54% of theory) of ethylgeranonitrile in the form of a mixture of double-bond isomers.

1.-15. (canceled)

16. A method of producing 3,7-dimethyl-2,6-nonadienenitrile comprising the steps
a) reacting a nitrile of the formula (II) 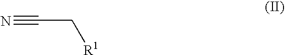
in which the radical
R¹ is hydrogen,
with a base as activation reagent to give the nitrile of the formula (II) in deprotonated form and
b) reacting the nitrile of the formula (II) in deprotonated form with 6-methyl-5-octen-2-one to give 3,7-dimethyl-2,6-nonadienenitrile.

17. The method according to claim 16 of producing 3,7-dimethyl-2,6-nonadienenitrile in the form of a mixture of configuration isomers and constitution isomers with regard to the ethylenic double bonds.

18. The method according to claim 16, which is carried out in acetonitrile as solvent.

19. The method according to claim 16, wherein a base with a pKa value of from 12 to 50 is used.

20. The method according to claim 16, wherein the reaction mixture obtained according to step a) is initially introduced and 6-methyl-5-octen-2-one is metered in.

21. A mixture comprising 3,7-dimethyl-2,6-nonadienenitrile and at least one compound selected from the group consisting of 3,7-dimethyl-3,6-nonadienenitrile and 3-methylene-7-methyl-6-nonenenitrile, where the content of 3,7-dimethyl-3,6-nonadienenitrile and 3-methylene-7-methyl-6-nonenenitrile together, based on the amount of 3,7-dimethyl-2,6-nonadienenitrile in the mixture, is up to 10% by weight.

22. The mixture according to claim 21 which consists of at least 90%, of 3,7-dimethyl-2,6-nonadienenitrile and at least one compound selected from the group consisting of 3,7-dimethyl-3,6-nonadienenitrile and 3-methylene-7-methyl-6-nonenenitrile.

23. A consumer article or everyday commodity comprising an organoleptically effective amount of the mixtures according to either of claim 21.

24. A fragrance composition comprising a mixture according to claim 21 and at least one further component selected from the group of fragrances consisting of 3,7-dimethyloctanenitrile and 3,7-dimethyl-6-octenenitrile.

25. The fragrance composition according to claim 24 comprising, in each case based on the total weight of the finished fragrance composition, 5 to 10% by weight of 3,7-dimethyloctanenitrile, 35 to 45% by weight of 3,7-dimethyl-6-octenenitrile and 50 to 60% by weight of 3,7-dimethyl-2,6-nonadienenitrile.

26. The fragrance composition according to claim 24 comprising ethylgeranonitrile in the form of a mixture comprising 3,7-dimethyl-2,6-nonadienenitrile and at least one compound selected from the group consisting of 3,7-dimethyl-3,6-nonadienenitrile and 3-methylene-7-methyl-6-nonenenitrile, where the content of 3,7-dimethyl-3,6-nonadienenitrile and 3-methylene-7-methyl-6-nonenenitrile together, based on the amount of 3,7-dimethyl-2,6-nonadienenitrile in the mixture, is up to 10% by weight.

27. The fragrance composition according to claim 24 comprising ethylgeranonitrile in the form of a mixture consists of at least 90%, of 3,7-dimethyl-2,6-nonadienenitrile and at least one compound selected from the group consisting of 3,7-dimethyl-3,6-nonadienenitrile and 3-methylene-7-methyl-6-nonenenitrile.

28. A process for producing a citrus odor which comprises utilizing the fragrance composition according to claim 24.

29. A consumer article or everyday commodity comprising an organoleptically effective amount of the fragrance compositions according to claim 24.

30. A consumer article or everyday commodity comprising an organoleptically effective amount of the fragrance compositions according to claim 27.
